(19) United States
(12) Patent Application Publication
(10) Pub. No.: US 2017/0158711 A1
(43) Pub. Date: Jun. 8, 2017

(54) NOVEL INDUSTRIAL CRYSTALLIZATION METHOD OF CEFUROXIME SODIUM AND PREPARATION THEREOF

(71) Applicants: HAINAN LINGKANG PHARMACEUTICAL CO., LTD., Haikou (CN); TIANJIN UNIVERSITY, Tianjin (CN)

(72) Inventors: Linggang TAO, Haikou (CN); Hongxun HAO, Tianjin (CN); Jingkang WANG, Tianjin (CN)

(73) Assignees: HAINAN LINGKANG PHARMACEUTICAL CO., LTD., Haikou (CN); TIANJIN UNIVERSITY, Tianjin (CN)

(21) Appl. No.: 15/306,084

(22) PCT Filed: Nov. 27, 2015
(86) PCT No.: PCT/CN2015/095810

§ 371 Date: Oct. 22, 2016

(30) Foreign Application Priority Data
Jun. 15, 2015 (CN) 201510330842.8

(51) Int. Cl. C07D 501/12; B01D 9/00; B01D 11/04

(52) U.S. Cl. CPC C07D 501/12; B01D 11/0411; B01D 9/0054; B01D 2009/0095

(57) ABSTRACT

It discloses a new industrial crystallization method of Cefuroxime Sodium, wherein supercritical fluid extraction technology and traditional crystalline technology are combined to realize the recrystallization of Cefuroxime Sodium. Processes such as extraction, adsorption, crystallization and drying are carried out with a supercritical fluid, a solvent, an extraction cell and a crystallization tank to realize the recrystallization of Cefuroxime Sodium under a specific pressure at a specific temperature.

This application is the U.S. national phase of International Application No. PCT/CN2015/095810 filed on 27 Nov. 2015 which designated the U.S. and claims priority to Chinese Application Nos. CN201510330842.8 filed on 15 Jun. 2015, the entire contents of each of which are hereby incorporated by reference.

### FIELD OF THE INVENTION

The invention relates to a novel industrial crystallization technology of Cefuroxime Sodium, and belongs to the technical field of medicine.

### PRIOR ART

Cefuroxime Sodium is also referred to Cefuroxime, and has a chemical name of Sodium (6R,7R)-7-[2-furyl (methoxyimino) acetylamino]-3-carbamoyloxymethyl-8-oxo-5-thia-1-azabicyclo [4.2.0]oct-2-ene-2-carboxylate. The molecular weight is 446.36 with a formula of C₁₆F₁₅N₄NaO₈S, and the chemical structure formula is shown as follows:

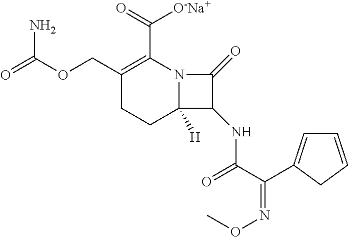

Cefuroxime Sodium is a white, off-white or yellowish powder or crystalline powder, odorless, bitter taste, and has cited moist. This product is soluble in water, slightly soluble in methanol, and insoluble in ethanol or chloroform. The specific rotation is +55° to +65° in 1 mL solution of 10 mg Cefuroxime Sodium, and the absorption coefficient (E1 cm1%) is 390^{˜}425 determined at the wavelength of 274 nm by spectrophotometry.

Cefuroxime Sodium is the best second-generation cephalosporin possessing advantages of the first-generation and the third-generation cephalosporins, and has strong antibacterial activities not only against Gram-positive bacteria but also against some Gram-negative bacteria, and it is a preferred drug especially in the treatment of Gram-positive and Gram-negative bacterial mixed infections. Due to broad spectrum antibiotic, wide distribution in vivo, high tissue concentration and low toxicity, Cefuroxime Sodium is applicable to respiratory infections, urinary tract infections, ear nose and throat infections, skin and soft tissue infections, obstetrics and gynecology infections, gonorrhea, septicemia, meningitis, internal and external surgical infections, and so on. Cefuroxime Sodium is not only used for anti-infective therapy in surgery, but also has obvious effects on anti-infective therapy after surgery, and prevention of surgical infection. Cefuroxime Sodium is not metabolized by the liver in vivo, which shows non-toxic to the liver, and excreting prototype from urine by the kidneys indicates almost non-toxic side effects to the kidney, therefore, it is a very safe drug and has good pharmacokinetic and security to the newborns. The above-mentioned advantages of the medicine make it a preferred drug for treating Gram-negative bacteria infections and Gram-negative and Gram-positive bacteria mixed infections.

The initial preparation route of Cefuroxime Sodium is proposed by British Glax Company, and it is prepared by 7-aminocephalosporanic acid through eight steps. Mainly due to the introducing of protecting groups for amino and carboxyl in the middle steps and the final deprotection, the yield is low with many impurities in product. Subsequently, many other preparation methods are provided, for example, in which 7-ACA firstly reacts with SMIF-C1 to form 7-[(z)-2-furanyl-2-methoxy imino acetamido]-3-acetoxymethyl-3-cephalosporanic acid (7-FCA), which is hydrolyzed by sodium hydroxide to produce 7-FHCA, then the later reacts with trichloroacetyl isocyanate to give cefuroxime acid, and finally cefuroxime acid is converted to Cefuroxime Sodium through a salt formation process. This method has higher hydrolysis yield, but if the chloride activity is too high, it will prone to generate side effects, and the product has darker color. In addition, sealed refrigeration at 2^{˜}8° C. is needed due to the poor stability of Cefuroxime Sodium, improper storage or transportation will deepen the color of the solid, and the color of the solution is often unqualified when it is tested in accordance with pharmacopoeia standards.

Due to the influence of the synthetic process of the raw material and properties of the drug itself, Cefuroxime Sodium currently used in clinical has serious problems like quality instability, bad color and so on. Thus the quality of product is influenced, causing the formulation not clarifies and turbidity unqualified, and the stability of the formulation is reduced.

Recrystallization process of Cefuroxime Sodium has been reported by numerous documents, such as UK Patent GB 2,012,270, Chinese patent CN 101,054,386, and Chinese patent CN 101,967,156. However, all these methods use traditional solventing-out crystallization with complex operations and tedious post-processing, easy to introduce new impurities, and severely limited in large-scale production.

Therefore, there is an urgent need to find a better solution to solve the problems of the prior art.

### DETAILED DESCRIPTION OF THE INVENTION

The aim of the present invention is to solve the problems of impurities and dark color in existing Cefuroxime Sodium, to simplify processes, improve efficiency, and provide an alternative crystallization method and apparatus of Cefuroxime Sodium for industrialization. Cefuroxime Sodium refined by this technology and apparatus has a color meeting the quality requirement, and possesses high product quality, good stability, and high-speed dissolution. Meanwhile, the present invention also provides Cefuroxime Sodium prepared by this technology and apparatus, and a sterile powder for injection containing Cefuroxime Sodium.

In the present invention, the essence of the technology used to refine Cefuroxime Sodium is a method to prepare product with high purity from the crude Cefuroxime Sodium, and the purity can exceed 99% after primary crystallization.

The technical solution of the present invention is based on the principle of the supercritical fluid extraction technology and the traditional crystallization technology. Firstly, dissolving Cefuroxime Sodium into a solution in a dissolving tank, and then extracting the organic solvent and the dissolved impurities in Cefuroxime Sodium solution by a supercritical fluid. By adjusting the pressure and the temperature, Cefuroxime Sodium is crystallized and separated from its solution in the dissolving tank.

The present invention is characterized in that extracting the organic solvent and the solute in a mixed system by the supercritical fluid, and changing the solubility of the ingredients in the organic solvent and the supercritical fluid, so as to make the solute crystallize. Thus a one-time crystallization of the active substance is realized and the product with high purity is obtained.

The present invention combines the technologies of extraction, adsorption, crystallization, and drying, and has advantages of high separation efficiency, non-toxic solvent residue, not easily degraded active ingredient, and so on.

In the technical solution of the present invention, the extraction is carried out for 5^{˜}20 minutes under a pressure of 15^{˜}40 MPa at a temperature of 40^{˜}60° C., and the crystallization is carried out for 20^{˜}40 minutes under a pressure of 0.5^{˜}5 MPa at a temperature of 20^{˜}30° C.

The process schematic of the method is shown in the Figure.

As shown in the Figure, the devices mainly include a working medium cylinder, a compressor, a heat exchanger, an extraction cell, a crystallization tank, and so on.

As shown in the Figure, the supercritical fluid is formed after pressurizing the work medium. The working medium can be CO₂, alkanes, alkenes, and so on, and CO₂ is preferable.

The solvent used to dissolve Cefuroxime Sodium is selected from one of alcohols, aldehydes, esters, ketones, ethers and, water and so on or a mixture thereof.

The selected solvent has a greater distribution coefficient in the supercritical fluid CO₂ than that of Cefuroxime Sodium. An aqueous ethanol is preferable, and 50%^{˜}80% aqueous ethanol is more preferable.

The extraction cell is used to form a mixed system comprising a solvent, a working medium and Cefuroxime Sodium by pressing. The surface of the extraction cell is coated with activated carbon, macroporous absorption resin or other materials to enhance the adsorption and selectivity of the impurities in the solution.

The crystallization tank is used for the separation of the solvent, the working medium and extracted Cefuroxime Sodium under reduced pressure.

A freely opened and closed fast interface with an internal filter capable of sterilization is set between the extraction cell and the crystallization tank.

When the system is working and the extraction cell and the crystallization tank are all at their respective temperatures and pressures, the supercritical fluid and the solution of Cefuroxime Sodium are extracted and adsorbed in the extraction cell, and the mixed system realizes crystallization and distillation in the crystallization tank. After the system is cooled and a pressure balance is arrived, Cefuroxime Sodium with high purity can be collected from the crystallization tank.

In a preferred embodiment, the present invention provides a crystallization method of Cefuroxime Sodium, which comprises the following steps:

(1) Crude Cefuroxime Sodium is weighed and placed to the extraction cell, adding a mixed solvent of 50%^{˜}80% aqueous ethanol, stirring until Cefuroxime Sodium is dissolved at a temperature of 40^{˜}60° C.;

(2) Pumping CO₂ fluid to 15^{˜}40 MPa by a high pressure liquid pump, stirring and maintaining the pressure and the temperature for 5 to 20 minutes, and then turning off the high pressure pump;

(3) Adding seed crystal to the crystallization tank, and lifting the height of the extraction cell up to 30 cm, thereafter opening the fast interface between the two cells, so that the liquid in the extraction cell enters the crystallization tank, and then closing the fast interface;

(4) Adjusting the pressure of the crystallization tank to 0.5^{˜}5 MPa and the temperature to 20^{˜}30° C., maintaining the temperature and the pressure for 20^{˜}40 minutes;

(5) After the system is cooled down and the pressure is dropped, a crystalline of Cefuroxime Sodium with high purity is prepared by drying under reduced pressure.

The solvent used for the crystallization separation of this technology is a supercritical fluid, and the supercritical fluid extraction technology is combined with the traditional crystallization separation technology to set extraction, adsorption, crystallization, and drying together. Under the combined effects of the supercritical fluid, solvents, the extraction cell and the crystallization tank, further crystallization and refining of Cefuroxime Sodium is realized with high purity and high yield, and the processes of enrichment and crystallization are also greatly simplified.

There are obvious differences between the new industrial crystallization technology of the present invention and traditional solventing-out recrystallization method. At same temperature, the crystallization method of the present invention takes shorter time and has higher crystallization efficiency. The product obtained from the primary crystallization of the present invention also has higher purity. The process of this method is simple without complicated energy-consumption and time-consuming processes such as column chromatography operation. Meanwhile, the yield of Cefuroxime Sodium in this method is higher than those of traditional processes, the purity exceeds 99% and the crystallization efficiency is greater than 80% after the primary crystallization of crude Cefuroxime Sodium, thus the method is suitable for production in large scale.

Crystallization efficiency of Cefuroxime Sodium (%)=[Weight of crystalline product (g)*content (%)]/[Weight of feedstock (g)*content (%)]*100%.

In the present invention, the novel industrial crystallization technology for refining Cefuroxime Sodium has solved the problems of impurities, dark color and poor stability in existing Cefuroxime Sodium. The obtained Cefuroxime Sodium meets the requirements of injection, and can be used to prepare a sterile powder for injection.

### BRIEF DESCRIPTION OF THE DRAWINGS

Hereinafter, the embodiment of the present invention is described in detail combined with the drawing, wherein:

FIG. 1 shows the schematic diagram of the apparatus used in the present method, wherein, 1 represents a thermostatically controlled heater, 2 represents an extraction cell, 3 represents a crystallization tank, 4 represents a stirrer, 5 represents a sensor, 6 represents a digital monitor, 7 represents a fast interface, 8 represents a cooling system, 9 represents a high pressure pump, 10 represents a cylinder, 11 represents a gas collector, and 12 represents a polystyrene insulator.

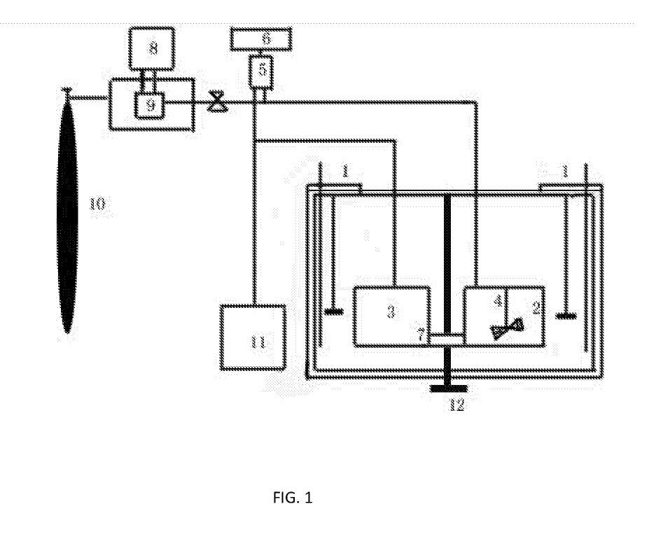

### EMBODIMENTS OF THE INVENTION

The present invention is further illustrated by the following embodiments without limiting the scope of the present invention. The present invention is further illustrated referring to the following examples, and a person skilled in the art should appreciate that the present invention is not limited to the embodiments and preparation methods. Moreover, a person skilled in the art is permitted making equivalent replacements, combination, improvement or modification according to the description of the present invention, but these all fall into the scope of the present invention.

The method for detecting the purity of Cefuroxime Sodium is as follows:

HPLC is used for detecting the purity of Cefuroxime Sodium, and the chromatographic condition includes:

Filler: octyl silane bonded silica gel;

Mobile phase: pH 3.4 acetate buffer (weighing 0.68 g sodium acetate and 5.8 g acetic acid, diluting to 1000 ml with water, and adjusting the pH value to 3.4 with acetic acid)—acetonitrile (85:15);

Detection wavelength: 273 nm;

Injection volume: 20 μl.

### Example 1

(1) 5.43 kg crude Cefuroxime Sodium with a purity of 93.4% was weighed and placed in the extraction cell, adding a mixed solvent of 50 kg 50% aqueous ethanol, and stirring until Cefuroxime Sodium dissolved at a temperature of 40° C.;

(2) Pumping CO₂ fluid to 15 MPa by a high pressure liquid pump, stirring and maintaining the pressure and the temperature for 5 minutes, and then turning off the high pressure pump;

(3) Adding seed crystal to the crystallization tank, lifting the height of the extraction cell to 30 cm, thereafter opening the fast interface between the two cells, so that the liquid in the extraction cell entered the crystallization tank, and closing the fast interface;

(4) Adjusting the pressure of the crystallization tank to 0.5 MPa and the temperature to 20° C., maintaining the temperature and the pressure for 20 minutes;

(5) After the system was cooled down and the pressure was dropped, 4.52 kg crystalline of Cefuroxime Sodium with high purity was prepared by drying under reduced pressure, and a sterile powder of Cefuroxime Sodium was obtained after sterile packing;

(6) As determined by HPLC, the Cefuroxime Sodium had a purity of 99.5% with a crystallization ratio of 88.7%.

### Example 2

(1) 5.66 kg crude Cefuroxime Sodium with a purity of 93.4% was weighed and placed in the extraction cell, adding a mixed solvent of 60 kg 80% aqueous ethanol, and stirring until Cefuroxime Sodium dissolved at a temperature of 60° C.;

(2) Pumping CO₂ fluid to 40 MPa by a high pressure liquid pump, stirring and maintaining the pressure and the temperature for 20 minutes, and then turning off the high pressure pump;

(3) Adding seed crystal to the crystallization tank, lifting the height of the extraction cell to 30 cm, thereafter opening the fast interface between the two cells, so that the liquid in the extraction cell entered the crystallization tank, and closing the fast interface;

(4) Adjusting the pressure of the crystallization tank to 5 MPa and the temperature to 30° C., and maintaining the temperature and the pressure for 40 minutes;

(5) After the system was cooled down and the pressure was dropped, 4.66 kg crystalline of Cefuroxime Sodium with a high purity was prepared by drying under reduced pressure and a sterile powder of Cefuroxime Sodium was obtained after sterile packing;

(6) As determined by HPLC, the Cefuroxime Sodium had a purity of 99.6% with a crystallization ratio of 87.8%.

### Example 3

(1) 6.97 kg crude Cefuroxime Sodium with a purity of 93.4% was weighed and placed in the extraction cell, adding a mixed solvent of 70 kg 70% aqueous ethanol, and stirring until Cefuroxime Sodium dissolved at a temperature of 50° C.;

(2) Pumping CO₂ fluid to 30 MPa by a high pressure liquid pump, stirring and maintaining the pressure and the temperature for 10 minutes, and then turning off the high pressure pump;

(3) Adding seed crystal to the crystallization tank, lifting the height of the extraction cell to 30 cm, thereafter opening the fast interface between the two cells, so that the liquid in the extraction cell entered the crystallization tank, and closing the fast interface;

(4) Adjusting the pressure of the crystallization tank to 1 MPa and the temperature to 25° C., and maintaining the temperature and the pressure for 30 minutes;

(5) After the system was cooled down and the pressure was dropped, 5.65 kg crystalline of Cefuroxime Sodium with a high purity was prepared by drying under reduced pressure and a sterile powder of Cefuroxime Sodium was obtained after sterile packing;

(6) As determined by HPLC, the Cefuroxime Sodium had a purity of 99.9% with a crystallization ratio of 86.7%.

### Example 4

(1) 4.47 kg crude Cefuroxime Sodium with a purity of 93.4% was weighed and placed in the extraction cell, adding a mixed solvent of 50 kg 75% aqueous ethanol, and stirring until Cefuroxime Sodium dissolved at a temperature of 55° C.;

(2) Pumping CO₂ fluid to 20 MPa by a high pressure liquid pump, stirring and maintaining the pressure and the temperature for 15 minutes, and then turning off the high pressure pump;

(3) Adding seed crystal to the crystallization tank, lifting the height of the extraction cell to 25 cm, thereafter opening the fast interface between the two cells, so that the liquid in the extraction cell entered the crystallization tank, and closing the fast interface;

(4) Adjusting the pressure of the crystallization tank to 4 MPa and the temperature to 25° C., and maintaining the temperature and the pressure for 35 minutes;

(5) After the system was cooled down and the pressure was dropped, 3.85 kg crystalline of Cefuroxime Sodium with a high purity was prepared by drying under reduced pressure and a sterile powder of Cefuroxime Sodium was obtained after sterile packing;

(6) As determined by HPLC, the Cefuroxime Sodium had a purity of 99.7% with a crystallization ratio of 91.9%.

### Comparative Example 1

(1) 1.44 kg crude Cefuroxime Sodium with a purity of 93.4% was weighed and placed in the reactor, adding 20 kg water, and stirring until Cefuroxime Sodium dissolved at a temperature of 60° C.;

(2) Adding acetone to the above solution, while cooling down to room temperature and standing for 6 hours;

(3) 0.67 kg Cefuroxime Sodium was obtained by drying under reduced pressure;

(4) As determined by HPLC, the Cefuroxime Sodium had a purity of 95.2% with a crystallization ratio of 47.4%.

### Experimental Example 1

In order to examine the flowability of the refined Cefuroxime Sodium prepared in Example 1, the angle of repose was measured by a funnel method.

Test method: The particles were placed in a fixed funnel, and were felled freely to a horizontal plane to form a disk-shaped stacked body with a bottom radius of r, and the height of the stacked body was measured as H. The results were calculated according to the equation of tan θ=H/r and were shown in the following table:

TABLE 1
Flowability test results of Cefuroxime Sodium

| Test items | Height H | Radius r | Angle of repose θ |
|---|---|---|---|
| The first time | 30 mm | 61.5 mm | 25.9° |
| The second time | 30 mm | 61.2 mm | 26.1° |
| The third time | 30 mm | 60.9 mm | 26.2° |
| Average value | 30 mm | 61.3 mm | 26.1° |

Conclusion: In general, less than 30 of the angle of repose indicates a good flowability of powders or granules. The angle of repose θ of above described crystalline powder of Cefuroxime Sodium is 26.1% which is less than 30°, suggesting it has a good flowability and is suitable for packing as a sterile powder for injection.

### Experimental Example 2

Referring to the quality standard of the raw material in “Part 2 of Chinese Pharmacopoeia 2010 Edition”, the quality research on crystalline powder of Cefuroxime Sodium was conducted in Example 1^{˜}4 and Comparative Example 1, and the results were shown as follows:

TABLE 2
Quality results of Cefuroxime Sodium

| | | | | | Comparative |
|---|---|---|---|---|---|
| Test items | Example 1 | Example 2 | Example 3 | Example 4 | Example 1 |
| Appearance | White | White | White | White | Pale yellow |
| | crystalline | crystalline | crystalline | crystalline | crystalline |
| | powder | powder | powder | powder | powder |
| pH | pH = 7.3 | pH = 7.5 | pH = 7.4 | pH = 7.0 | pH = 7.5 |
| Clarity of the | Compliance | Compliance | Compliance | Compliance | Compliance |
| solution | | | | | |
| Color of the | Compliance | Compliance | Compliance | Compliance | Greater than |
| solution | | | | | yellow No. 6 |
| Related | Single | Single | Single | Single | Single |
| substances | impurity: 0.046% | impurity: 0.051% | impurity: 0.065% | impurity: 0.057% | impurity: 1.22% |
| | Total | Total | Total | Total | Total |
| | impurity: 0.101% | impurity: 0.096% | impurity: 0.114% | impurity: 0.095% | impurity: 3.45% |
| Cefuroxime polymer | 0.031% | 0.012% | 0.022% | 0.019% | 0.24% |
| Solvent residue | Compliance | Compliance | Compliance | Compliance | Compliance |
| 2-ethyl | 0.024% | 0.078% | 0.039% | 0.096% | 0.258% |
| hexanoic acid | | | | | |
| Water | 1.16% | 0.99% | 1.42% | 0.96% | 0.99% |
| Visible foreign | Compliance | Compliance | Compliance | Compliance | Compliance |
| matter | | | | | |
| Insoluble | Compliance | Compliance | Compliance | Compliance | Compliance |
| particles | | | | | |
| Heavy metal | Compliance | Compliance | Compliance | Compliance | Compliance |
| Abnormal | Compliance | Compliance | Compliance | Compliance | Compliance |
| toxicity | | | | | |
| Bacterial | Compliance | Compliance | Compliance | Compliance | Compliance |
| endotoxin | | | | | |
| Content | 94.6% | 94.7% | 95.0% | 94.8% | 90.5% |
| (counted on | | | | | |
| Cefuroxime) | | | | | |

Conclusion: Each test items of the crystalline powder of Cefuroxime Sodium in Example 1^{˜}4 was in line with the regulation, while the color of the solution, related substances, cefuroxime polymer and content in Comparative Example 1 didn't comply with the regulation, Therefore, the crystalline powder of Cefuroxime Sodium prepared by this technology met the quality requirements of “Part 2 of Chinese Pharmacopoeia 2010 edition”.

Industrial Practicability

What can see from the results of the examples and experimental examples described above, the crystalline powder of Cefuroxime Sodium prepared by the new industrial crystallization technology and apparatus of the present invention has high yield and high purity, with each index in line with the regulation. The crystalline powder of Cefuroxime Sodium is suitable for preparing as sterile powder for injection, and possessing good value in industry.

The present invention has been described above in detail through the embodiments and examples. However, it should be understood that the description does not make any restrictions to the scope of the present invention. Without departing from the sprit and scope of the present invention, various modifications, improvement, and replacements is permitted, and will be seen in the scope of the present invention.

What is claimed is:

1. A novel industrial crystallization method of Cefuroxime Sodium, characterized in that, combining supercritical fluid extraction and traditional crystallization to realize the recrystallization of Cefuroxime Sodium, preferably dissolving Cefuroxime Sodium in an organic solvent, extracting the organic solvent and the solute in the solution of Cefuroxime Sodium by a supercritical fluid, and changing the solubility of the ingredients in the organic solvent and the supercritical fluid by adjusting the temperature and the pressure to make Cefuroxime Sodium crystallize.

2. The method according to claim 1, characterized in that including Cefuroxime Sodium prepared by the method, and a sterile powder for injection containing Cefuroxime Sodium.

3. The method according to claim 2, characterized in that, the solution of Cefuroxime Sodium is subjected to supercritical fluid extraction for 5^{˜}20 minutes under a pressure of 15^{˜}40 MPa at a temperature of 40^{˜}60° C., and Cefuroxime Sodium is crystallized and separated from its solution for 20^{˜}40 minutes under a pressure of 0.5^{˜}5 MPa at a temperature of 20^{˜}30° C.

4. The method according to claim 3, characterized in that, said solvent for dissolving Cefuroxime Sodium is selected from one of alcohols, aldehydes, esters, ketones, ethers, water and so on or a mixture thereof, an aqueous ethanol is preferable, and an aqueous ethanol with a concentration of 50%^{˜}80% is more preferable.

5. The method according to claim 4, characterized in that, the working medium for forming the supercritical fluid can be CO₂, alkanes, alkenes and so on, and CO₂ is preferable.

6. The method according to claim 5, characterized in that, devices, such as a working medium cylinder, a compressor, a heat exchanger, a dissolving tank, and a crystallization tank, are used.

7. The method according to claim 6, characterized in that, the surface of the dissolving tank is coated with activated carbon, macroporous absorption resin, and so on, a stirring device is set in the extraction cell, and a freely opened and closed fast interface with a internal filter capable of sterilization is set between the dissolving tank and the crystallization tank.
